(19) United States
(12) Patent Application Publication
(10) Pub. No.: US 2004/0053898 A1
(43) Pub. Date: Mar. 18, 2004

(54) Combination preparation with a erbeta selective estrogen and serm or antiestorgen

(76) Inventors: Karl-Heinrich Fritzemeier, Berlin (DE); Uwe Kollenkirchen, Berlin (DE); Christa Hegele-Hartung, Muelheim a.d. Ruhr (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/344,161

(22) PCT Filed: Aug. 3, 2001
(86) PCT No.: PCT/EP01/09008

(30) Foreign Application Priority Data
Aug. 10, 2000 (DE) 100 39 199.0

(51) Int. Cl. A61K 31/57; A61K 31/56

(52) U.S. Cl. 514/170

(57) ABSTRACT

A novel medicament for the treatment of estrogen-deficient disease states is disclosed. Said medicament is a combination preparation comprising an ERβ-selective estrogen and an ERα-selective antiestrogen or SERM (Selective Estrogen Receptor Modulator). The antiestrogen or SERM which is a component of the combination preparation is preferably selective for the periphery. The preparation is suitable for an organ-specific estrogen therapy and has clear advantages over conventional therapies. Due to the combination of ERα-selective SERM and ERβ-estrogen the preparation permits a complete protection against bone loss caused by estrogen deficiency. The components of the medicament also have a synergistic effect with respect to the inhibition of inflammation inducing genes, in particular in inflammatory disorders such as atherosclerosis and arthritis, or neurodegenerative diseases such as Alzheimers and multiple sclerosis. Furthermore, positive effects on cognition and mood may be expected. The protective estrogen-like effects are achieved, with no expectation of proliferation effects on breasts or uterus.

### DESCRIPTION

This invention relates to a combination preparation that comprises an ERβ-specific agonist and an antiestrogen or SERM, preferably an ERα-selective antiestrogen, in particular a peripherally selective ERα-selective antiestrogen and/or an ERα-selective SERM.

The efficiency of estrogens for treatment of hormone-deficiency-induced symptoms such as hot flashes and atrophy of estrogen target organs, as well as for preventing bone mass loss in perimenopausal women and postmenopausal women is readily confirmed and generally accepted. It is also well-documented that the estrogen replacement therapy in postmenopausal women or in women with ovarian dysfunction that is caused in some other way reduces the risk of cardiovascular diseases compared to non-estrogen-treated women (Grady et al. (1992), Ann Intern Med 117, 1016-1037).

More recent studies confirm, moreover, a protective action of estrogens against neurodegenerative diseases, such as, e.g., Alzheimer's disease (Henderson (1997), Neurology 48 (Suppl 7), p. 27-p. 35; Birge (1997), Neurology 48 (Suppl 7), p. 36-p. 41), a protective action with respect to brain functions, such as memory and learning capacity (McEwen et al. (1997), Neurology 48 (Suppl 7), p. 8-p. 15; Sherwin (1997), Neurology 48 (Suppl 7), p. 21-p. 26), as well as against hormone-deficiency-induced mood swings (Halbreich (1997), Neurology 48 (Suppl 7), p. 16-p. 20).

In conventional estrogen or hormone replacement therapy, standard estrogens such as estradiol and conjugated estrogens that consist of equine urine are used either by themselves or in combination with a gestagen.

Because of the stimulating action of standard estrogens on the endometrium, which results in an increased risk of endometrial carcinoma (Harlap, S. (1992), Am J Obstet Gynecol 166, 1986-1992), primarily estrogen/gestagen combination preparations are used in hormone replacement therapy. The estrogen/gestagen combination avoids a hypertrophy of the endometrium, but the occurrence of undesirable intracyclic menstrual bleeding is also linked with the combination.

Estrogens, which are substances that have an estrogen-like effect on the brain, bones and vascular system but do not have a proliferative effect on the endometrium, represent an alternative to the estrogen/gestagen combination preparations.

A class of substances that partially fulfill the desired profile of a selective estrogen are the so-called selective estrogen receptor modulators (SERM) (R. F. Kauffmann, H. U. Bryant (1995), DN@P 8 (9), 531-539). In this case, these are partial agonists/partial antagonists of the estrogen receptor subtype ERα. These SERMs act on ERβ as pure antagonists (McInnerney et al. (1998), Endocrinol. 139, 4513-4522). Because of their antiestrogenic nature, SERMS are ineffective with respect to the therapy of acute postmenopausal symptoms, such as, e.g., hot flashes.

Estrogens exert their physiological action via a receptor protein, the estrogen receptor (ER). In this case, this is a nuclear-position transcription factor that can be activated by ligands. Until a few years ago, it was assumed that estrogens exert their action via a single receptor. Recently, however, ERβ was discovered as a second subtype of estrogen receptor (Kuiper et al. (1996), Proc. Natl. Acad. Sci. 93, 5925-5930; Mosselman, Dijkema (1996), Febs Letters 392, 49-53; Tremblay et al. (1997), Molecular Endocrinology 11, 353-365). The expression pattern of ERβ differs from that of ERα (Kuiper et al. (1996), Endocrinology 138, 863-870). ERβ thus predominates in the rat prostate over ERα (Chang, Prins (1999), The Prostate 40, 115-124), while ERα predominates in the rat uterus. In the brain, areas in which in each case only one of the two ER-subtypes is expressed were identified (Shugrue et al. (1996), Steroids 61, 678-681; Li et al. (1997), Neuroendocrinology 66, 63-67). ERβ is, i.a., expressed in areas that are considered to be important for cognitive processes and “mood” (Shugrue et al. (1997), J. Comparative Neurology 388, 507-525).

Other organs that predominantly express ERβ are the gastrointestinal tract (Campbell-Thomson (1997), Bioch. Biophys. Res. Com. 240, 478-483), the urogenital tract (Kuiper et al. (1996), Endocrinology 138, 863-870), the granulosa cells of the ovary (Byers et al. (1997), Mol. Endocrinol. 11, 172-182), and the myocardium (Gustafsson (Nice, September 1999), hearing). However, predominantly ERα is expressed in the liver, the kidney and the pituitary gland (Shugrue et al. (1998), Steroids 63, 498-504). In the uterus, ERα dominates (Wang et al. (1999), Biol. of Reprod. 61, 955-964).

In bones (Kuiper et al. (1998), Frontiers in Neuroendocrinology 19, 253-286) and blood vessels, both ERα and ERβ are expressed (Lafrati et al. (1997), Nature Med. 3, 545-48).

The ERs exert-their action as ligand-activated transcription factors. After binding of the hormone, receptor dimerization is carried out. Based on the expression of ERα and/or ERβ in a cell, a homodimer or heterodimer ERα and ERβ is formed (Cowley et al. (1997), J. Biol. Chem. 272, 19858-19862). The dimer binds to a specific sequence in the promoter of a target gene, the “estrogen response element” ERE (Kumar, Chambon (1988), Cell 55, 145-156; Klein-Hitpass et al. (1986), Cell 46, 1053-1061). Binding of the receptor dimer to the ERE produces the recruiting of essential transcription factors and the initiation of the transcription.

Interestingly enough, in cells that express both ERα and ERβ, the estradiol-induced transcription activation is reduced compared to cells that express only ERα. In such cells, ERβ acts as a repressor of ERα-stimulated transactivation (Hall, McDonnell (1999), Endocrinology 140, 5566-5578). It is attributed to this function of ERβ as a modulator of ERα with respect to transactivation that in ERβ-knock-out mice, the response to estrogen administration in the uterus is more strongly pronounced than in wild-type mice (Gustafsson, Steamboat Springs (February 2000), hearing).

In addition to the action of ER(s) as activators of transcripts, they exert control on the expression of other genes by inhibiting their activation by other transcription factors. It thus was shown that estrogens inhibit the expression of the cytokine interleukin-6 (IL-6) (Pottratz et al. (1993), J. Clin. Invest. 93, 944-950; Stein, Young (1995), Mol. Cell Biol. 15, 4971-4979). Other inflammation-induced genes are also inhibited by estrogens, such as, e.g., the COXII-expression in blood vessels of rats (Fritzemeier, Hegele-Hartung (1999), Handbook of Pharmacol., Oettel, Schillinger Editors, 135/II, 21, 1-94). IL-6 is considered as a central mediator of immune and inflammation reactions, as well as osteoclastogenesis (Sehgal (1992), Res. Immunol., 724-734; Jones (1994), Clin. Endocrinol. 40, 703-713). Estrogens suppress the IL-6 production by osteoblasts and stroma cells of the bone marrow. Since IL-6 stimulates the osteoclast recruitment and maturation, estrogens have an inhibitory effect on this process by inhibiting the IL-6 production. This inhibition of the IL-6 production is carried out by inhibiting the expression of the IL-6 gene (Pottratz et al. (1993), J. Clin. Invest. 93, 944-950; Stein, Young (1995), Mol. Cell. Biol. 15, 4971-4979). The ER-mediated inhibitory action of the estrogens is produced by inhibition of the activity of transcription factor NF_{K}B. This transcription factor is activated by inflammatory signals (Thanos, Maniatis (1995), Cell 80, 529-532; Didonato et al. (1997), Nature 388, 548-554). It is expected that the ER interacts directly with NF_{K}B and blocks its binding to the NF_{K}B binding site in the promoter of inflammation-induced genes, such as IL-6 (Ray et al. (1997), FEBS Lett. 409, 79-85).

An IL-6 reporter gene assay was described by Pottratz et al. (1993), supra. The ligand-activated ER inhibited the activity of a reporter gene, which contained the NF_{K}B-binding site of the IL-6 gene in the promoter, in various cell lines (Pottratz et al. (1993), J. Clin. Invest. 93, 944-950).

A disadvantage of previous estrogen therapies often exists in low organ selectivity. The basic object of this invention consists in developing preparations for an estrogen therapy in which the drawbacks of the prior art are at least partially eliminated.

It was found, surprisingly enough, that an organ-selective estrogen therapy is possible by the administration of a combination preparation, including one for ERβ-selective agonists and an antiestrogen or a selective estrogen receptor modulator (SERM). The combination preparation is suitable for therapy or prophylaxis of estrogen-deficiency-induced diseases. The two components of the preparation can be administered in a common dispensing form (e.g., a preparation with two components) or in respectively separate dispensing forms (two preparations with one component in each case).

The combination preparation according to the invention is extremely well suited for an organ-selective estrogen therapy and clearly has superiority over existing therapies.

In a preferred embodiment of the invention, an ERα-selective antagonist, in particular a peripherally-selective ERα-selective antagonist, is used as an antiestrogen. In another preferred embodiment, an ERα-selective SERM is used.

By the medication, an at least largely complete estrogen action on the organ systems or tissues, such as the bones, the vascular system, the brain functions and components of the immune system is achieved, while only marginal or no estrogenic action on organ systems such as the uterus, liver, mammary gland and pituitary gland is produced.

The new medication is superior to conventional estrogen or hormone replacement therapy with estrogens or estrogen/gestagen combinations through a reduced action on the uterus and the avoidance of bleeding. The medication is superior to monotherapy with a SERM or an ERβ ligand through a more complete protection from estrogen-deficiency-induced bone mass loss. The medication that is described here is superior to the combination of a “standard” estrogen such as estradiol with a pure antiestrogen through an improved “therapeutic window” (clear dissociation between bone-protective and uterus-stimulating dosages).

By the special combination of the ERβ-selective estrogen with the ERα-selective antiestrogen or SERM, it is achieved that in cells and organ systems that exclusively or predominantly express ERβ, such as, e.g., the brain, ERβ-dependent estrogenic actions are induced through the ERβ-selective estrogen components of the preparation. In the uterus, in which ERα dominates over ERβ, the ERα-selective SERM or antiestrogen and the ERβ-agonist have an antiproliferative action in the same direction. In organs, such as the bone, in which both ERα and ERβ are expressed, the ERα-selective SERM or antiestrogen and the ERβ estrogen have an additive action with respect to protection against estrogen-deficiency-induced bone mass loss. Also, the ERα-selective SERM or antiestrogen and the ERβ-agonist in the vascular system exert an antiproliferative and anti-inflammatory action in the same direction and thus have a synergistic action with respect to protection against vascular diseases such as arteriosclerosis.

The invention relates to a combination preparation, its production, therapeutic application and pharmaceutical dispensing forms, consisting of a novel selective estrogen, an ERβ-selective estrogen and an antiestrogen, preferably a so-called SERM (S. R. Kauffman; H. U. Bryant (1995), DN@P 8 (9), 531-539). Especially preferred is the combination of an ERβ-selective estrogen with a SERM or with an antiestrogen that has a higher affinity to the rat uterus receptor, in comparison to the rat prostate receptor or to the ERα in comparison to the ERβ, in particular those compounds that are peripherally-selectively active, i.e., that do not pass the blood-brain barriers. An example of an ERα-selective SERM is raloxifene (Barkhelm et al. (1998), Mol. Pharmacol. 54, 105-112), which is claimed for this application. Examples of peripherally selective antiestrogens are ZM 182780, 11β-fluoro-7α-(14,14,15,15,15-pentafluoro-6-methyl-10-thia-6-azapentadecyl)-estra-1,3,5(10)-triene-3,17β-diol and other 7α-alkyl-estratrienes (PCT/EP97/045517) and 11β-fluoro-7-(13,13,14,14,15,15,16,16,16-nonafluoro-6-methyl-6-azahexadecyl)-estra-1,3,5(10)-triene-3,17β-diol. An example of a peripherally selective SERM is 5-(4-{5-[(RS)-(4,4,5,5,5-pentafluoropentyl)sulfinyl]pentyloxy}phenyl)-6-phenyl-8,9-dihydro-7H-benzocyclohepten-2-ol. Peripherally-selective antiestrogens and SERMs can be components of the medication that is described here and are claimed for this application. Also, other SERMs, such as 14α,17α-ethano-11β-{4-[5-(2-pyridinemethylsulfonyl)pentyloxy]phenyl}-1,3,5(10)estratriene-3,17β-diol (11β-substituted steroids), TSE 424 and other 2-phenylindoles (American Home), EM 652, EM 800, CP 336156 (lasofoxifenes, Pfizer; Hua et al. (2000), Endocrinology 141, 1338-1344) can be components of the combination preparation and are claimed for this application.

As a component of the combination preparation, an ERβ-selective estrogen is the subject of this invention and is distinguished by higher affinity to the estrogen receptor of the rat prostate in comparison to the rat uterus or by higher affinity to ERβ in comparison to ERα. This comprises substances that were described in earlier patent applications: “ERβ-affine ent-steroids; 16-OH-steroids; Nor-steroids; 8-β-substituted steroids.” This application also comprises other selective estrogens that were described in various patent applications as possible components of the combination preparation: e.g., a) ASTRA, Novel Estrogens, WO97/08188, 9502921-1, PCT/SE96/01028; b) Sumitomo Chemical Co. Ltd., JP 11292872; c) Androstenediol and Prodrugs of Androstenediol; Pharmaceutical Compositions and Uses for Androstene 3β,17β-Diol, WO99/63973) and d) Phytoestrogens with Higher Affinity to ERβ in Comparison to ERα.

The ERβ-agonist is preferably selected from 3,16-dihydroxyestra-1,3,5(10)-triene derivatives, 8α-H, 9β-H, 10α-H, 13β-H, and 14β-H gonane derivatives, preferably derived from ent-13-alkylgonane, 8β-substituted estra-1,3,5(10)-triene derivatives and gona-1,3,5(10)-triene derivatives. Examples of especially preferred ERβ-antagonists are described in PCT/EP00/01073, DE 199 17 930.1, DE 199 41 105.1 and DE 100 19 167.3. Reference is made expressly to the disclosure of these documents, in particular to the general structural formulas and preferred individual compounds that are shown there.

A selective estrogenic action of the preparation according to the invention can be achieved based on the different tissue distribution of ERα and ERβ by subtype-specific ligands. Substances with a preference for ERβ compared to ERα in the in-vitro receptor binding test were described by Kuiper et al. (Kuiper et al. (1996), Endocrinology 138, 863-870). Then, i.a., the phytoestrogen genisteine and the DHEA-metabolite androstenediol have ERβ-selectivity. Other ERβ-selective estrogens were described in various patents: ERβ-affine ent-steroids; 16-OH steroids; Nor-steroids; 8-β-substituted steroids. This application claims other selective estrogens and prodrugs that were described in various patent applications as possible components of the preparation: a) ASTRA, Novel Estrogens, WO97/08188, 9502921-1, PCT/SE96/01028; b) Sumitomo Chemical Co. Ltd., JP 11292872; c) Androstenediol and Prodrugs of Androstenediol; Pharmaceutical Compositions and Uses for Androstene 3β,17β-Diol, WO99/63973); Phytoestrogens with Higher Affinity to ERβ in comparison to ERα, such as genisteine.

Westernlind et al. (1998) describe a differential action of 16α-hydroxyestrone on the bone, on the one hand, and reproductive organs of the female rat, on the other hand (Westerlind et al. (1998), J. Bone and Mineral Res 13, 1023-1031).

Our studies produced the fact that 16α-hydroxyestrone binds 3× better to the human estrogen receptor α (ERα) than to the human estrogen receptor β (ERβ). The RBA value of the substance in the rat prostate estrogen receptor is 5× better than the RBA value of the substance in the rat uterus estrogen receptor. The dissociation of the substance that is described by Westerlind can be attributed, according to our findings, to the preference thereof for ERβ in comparison to ERα. 16α-Hydroxyestrone is a possible component of the preparation that is described here and is claimed for this application.

The combination preparation according to the invention is especially suitable for a tissue-selective or organ-selective estrogen therapy; for example for the prophylaxis or treatment of perimenopausal and postmenopausal symptoms, for hormone substitution, for prophylaxis or treatment of hormone-deficiency-induced symptoms, in particular in ovarian dysfunction, for prophylaxis and treatment of hormone-deficiency-induced bone mass loss and osteoporosis, for prophylaxis and treatment of cardiovascular and vascular diseases, for prophylaxis and treatment of hormone-deficiency-induced and neurodegenerative diseases, for prophylaxis and treatment of hormone-deficiency-induced impairments of memory and learning capacity, and for prophylaxis and treatment of diseases of the immune system.

The new medication is especially suitable for the treatment of perimenopausal and postmenopausal symptoms, especially hot flashes, sleep disorders, irritability, mood swings, incontinence, vaginal atrophy and hormone-deficiency-induced mental diseases. The preparation is also suitable for hormone substitution and the therapy of hormone-deficiency-induced symptoms in surgical, medicinal or ovarian dysfunction that is caused in some other way.

In addition, the preparation can be used to prevent hormone-deficiency-induced bone mass loss and osteoporosis, to prevent cardiovascular diseases, in particular vascular diseases such as arteriosclerosis, and to prevent hormone-deficiency-induced neurodegenerative diseases such as Alzheimer's disease as well as hormone-deficiency-induced impairment of memory and learning capacity.

In addition, the preparation can be used for treating inflammatory diseases of the immune system, in particular autoimmune diseases, such as, e.g., rheumatoid arthritis.

The medication is suitable for therapy and prophylaxis of estrogen-deficiency-induced diseases both of women and men.

In men, the medication is especially suitable for the therapy of hormone-deficiency-induced bone mass loss and osteoporosis, for preventing cardiovascular diseases, in particular vascular diseases such as arteriosclerosis, and for preventing hormone-deficiency-induced neurodegenerative diseases, such as Alzheimer's disease as well as hormone-deficiency-induced impairment of memory and learning capacity and for therapy of prostate hyperplasia.

In addition, the medication can be used for treating inflammatory diseases and diseases of the immune system, in particular autoimmune diseases, such as, e.g., rheumatoid arthritis.

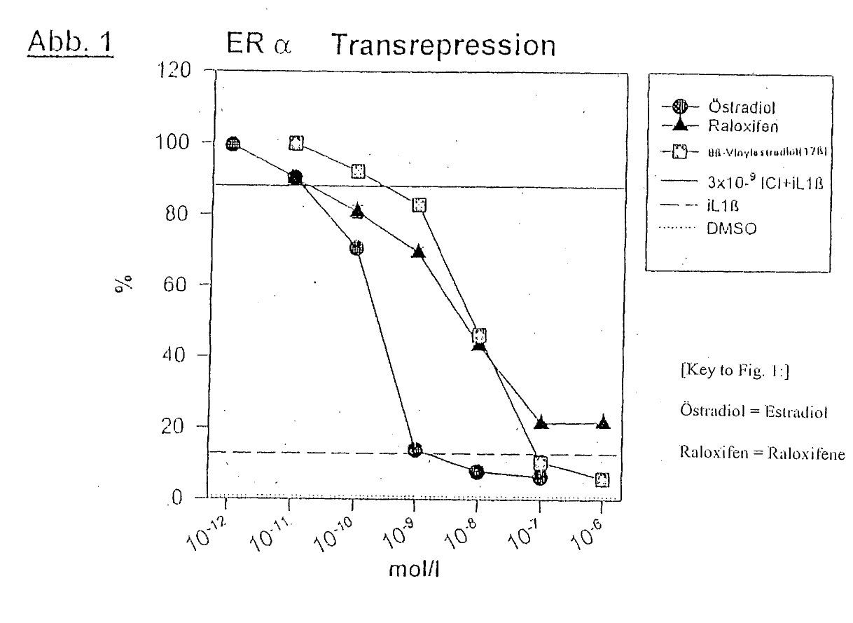

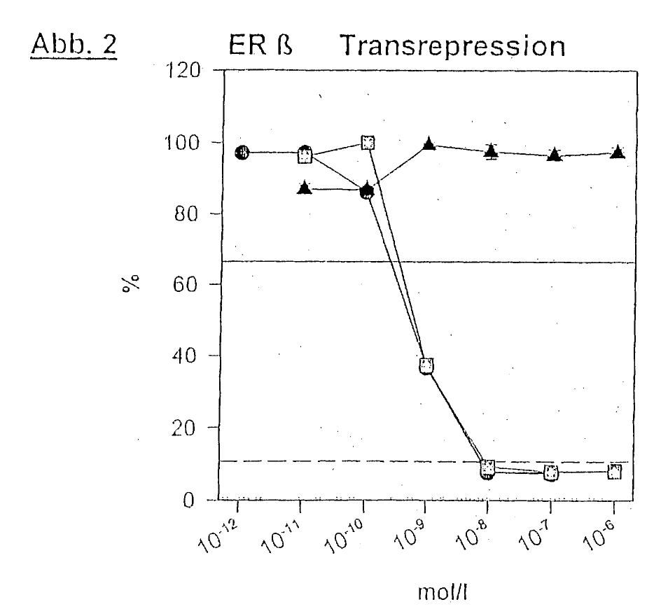

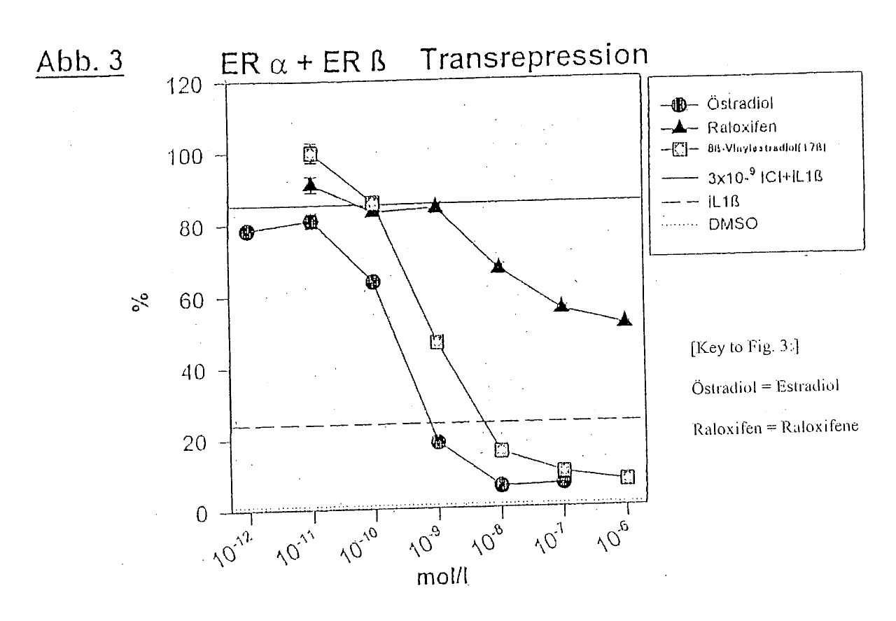

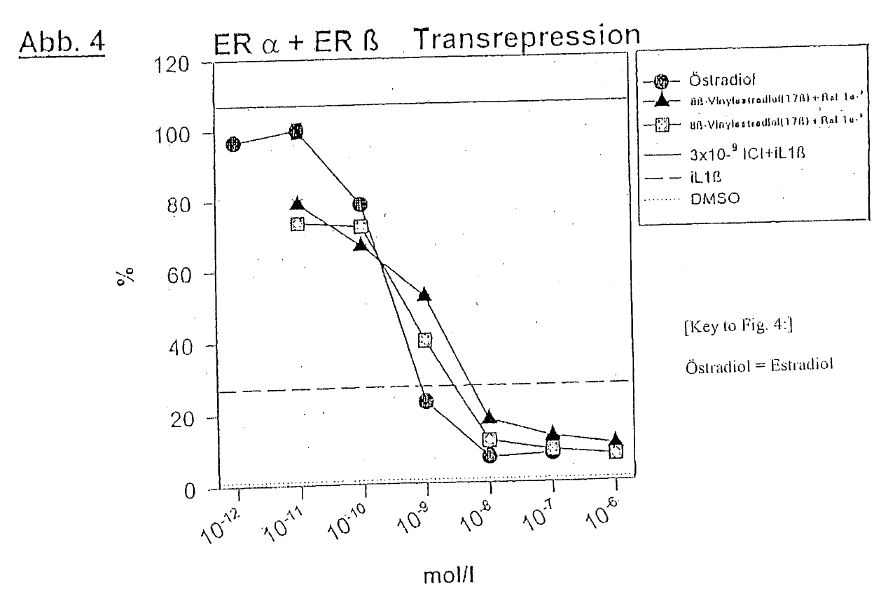

By the studies resulting in this invention, it was determined that ERβ is able to inhibit NF_{K}b-controlled reporter genes. In a reporter gene assay with an NF_{K}b-controlled reporter gene, the SERMs have thus proven their value as partial antagonists when they exert their action via ERα, i.e., they produce an estrogen-like inhibition of the reporter gene activity and exert an antagonistic (in terms of an active transrepressing) action in the presence of estradiol (FIG. 1). This action is reflected in vivo by an antiresorptive (bone-protective) action. If SERMs act via ERβ, however, they do not exert any agonistic action on an NF_{K}b-controlled reporter gene (FIG. 2). In co-transfection of ERα and ERβ, ERβ inhibits the ERα-mediated agonistic action of the SERMs (FIG. 3). Therefore, no complete protection against estrogen-deficiency-induced bone mass loss can be achieved by a SERM alone in vivo, since ERα and ERβ are expressed in the bone. Complete repression of the NF_{K}b-controlled promoter is achieved, however, surprisingly enough, by co-administration of a SERM and an ERβ-specific estrogen when ERα and ERβ are co-transfixed in the test cells (FIG. 4).

The additive action relative to an inhibition of the NF_{K}b-controlled promoter of SERM and ERβ-selective estrogen in cultivated cells, which express ERα and ERβ, involves an additive antiresorptive (bone-protective) action in vivo, since bone cells also express both ERα and ERβ in the intact organism. In addition, it can be arranged that the combination of ERβ-specific estrogen and SERM in vivo acts additively or synergistically relative to an inhibition of inflammation-induced genes, if the cells of the target organ express both ERα and ERβ. This holds true, e.g., for the cardiovascular system.

In addition, SERMs, in particular ERα-selective SERMs, allow a selective estrogen therapy in this respect since they inhibit the estrogen-deficiency-induced bone mass loss and in this case produce little or no stimulation of the uterus growth. Their bone-protective (antiresorptive) action is based on the inhibition of the expression of the osteoclast-stimulating cytokines. They exert this action via ERα in bone cells (inhibition of NF_{K}b). SERMs act on the uterus as antiestrogens; they inhibit estrogen-stimulated growth of the uterus, in particular the proliferation of the epithelium. They exert this action via ERα. SERMs also exert antiestrogen-and proliferation-inhibiting action on breast cancer cells. In addition, SERMs that are not peripherally selective show antiestrogenic action on estrogen-induced genes in the brain. In combination with an ERβ-selective agonist, this results in an organ-selective or tissue-selective action. Thus, for example, the protective estrogen-like actions are achieved without undesirable proliferative effects on the breast and uterus being expected.

The amounts of components (a) and (b) of the pharmaceutical combination preparation according to the invention that are to be administered can all be amounts with which the desired effects are achieved. Based on the condition to be treated and the type of administration, the amount of component (a) that is to be administered is preferably 0.01 μg/kg to 10 mg/kg of body weight, especially preferably 0.04 μg/kg to 1 mg/kg of body weight per day. In humans, this corresponds to, for example, a dose of 0.8 μg to 800 mg, preferably 3.2 μg to 80 mg daily. The amount of component (b) that is to be administered is preferably 0.01 μg/kg to 10 mg/kg of body weight, especially preferably 0.04 μg/kg to 1 mg/kg of body weight per day. A dosage unit of the pharmaceutical combination preparation according to the invention preferably contains 0.8 μg to 800 mg each, preferably 1.6 μg to 200 mg, of each of components (a) and (b).

The ratio of the two components (a) and (b) in the combination preparation according to the invention can vary over a wide range and is preferably 1:99 to 99:1 according to weight, especially preferably 10:90 to 20:10 according to weight. Based on the desired stimulation, it may be advantageous to select the amount of the active ingredients to be administered from the upper or lower range of the above-indicated amount ranges. As a result, the selectivity of the active ingredients can be further increased.

The administration of components (a) and (b) can be carried out simultaneously or in succession. It is possible in particular to administer the active ingredients alternately one after the other. Suitable administration protocols are, for example, subcutaneous administration or oral administration. The active ingredients can be administered several times daily, for example one to ten times daily, and over several days, for example over a period of 1 to 60 days, preferably from 1 to 30 days.

The pharmaceutical combination preparations contain the active ingredients optionally in a mixture with pharmacologically common vehicles, adjuvants or diluents, as well as optionally with other pharmacologically or pharmaceutically active substances, such as, for example, gestagens. The production of pharmaceutical agents is carried out in a known way.

As vehicles and adjuvants, e.g., those are suitable that are recommended or indicated in the following bibliographic references as adjuvants for pharmaceutics, cosmetics and related fields: Ullmanns Encyklopädie der technischen Chemie [Ullmann's Encyclopedia of Technical Chemistry], Volume 4 (195.3), pages 1 to 39; Journal of Pharmaceutical Sciences, Volume 52 (1963), pages 918 ff., issued by Czetsch-Lindenwald, Hilfsstoffe für Pharmazie und angrenzende Gebiete [Adjuvants for Pharmaceutics and Related Fields]; Pharm. Ind., No. 2 (1961), pages 72 and ff.: Dr. H. P. Fiedler, Lexikon der Hilfsstoffe für Pharmazie, Kosmetik und angrenzende Gebiete [Dictionary of Adjuvants for Pharmaceutics, Cosmetics and Related Fields], Cantor KG, Aulendorf in Württemberg 1971.

The compounds can be administered orally or parenterally, for example intraperitoneally, intramuscularly, subcutaneously or percutaneously. The compounds can also be implanted in the tissue.

For oral administration, capsules, pills, coated tablets, etc., are suitable. In addition to the active ingredient, the dosage units can contain a pharmaceutically compatible vehicle, such as, e.g., starch, sugar, sorbitol, gelatin, lubricant, silicic acid, talc, etc.

For parenteral administration, the active ingredients can be dissolved or suspended in a physiologically compatible diluent. As a diluent, very often oils are used with or without the addition of a solubilizer, a surfactant, a suspending agent or an emulsifier. Examples of oils that are used are olive oil, peanut oil, cottonseed oil, soybean oil, castor oil and sesame oil.

The compounds can also be used in the form of a depot injection or an implant preparation that can be formulated so that a delayed release of active ingredient is made possible.

Implants can contain, as inert materials, e.g., biodegradable polymers or synthetic silicones, such as, e.g., rubber gum. In addition, the active ingredients can be worked into, e.g., a plaster for percutaneous administration.

For the production of intravaginal rings (e.g., vaginal rings) or intrauterine systems (e.g., pessaries, coils, IUDs, Mirena®) that are charged with active ingredients for local administration, various polymers, such as, e.g., silicon polymers, ethylene vinyl acetate, polyethylene or polypropylene, are suitable.

To achieve a better bioavailability of the active ingredient, the compounds can also be formulated as cyclodextrin clathrates. To this end, the compounds are reacted with α-, β- or γ-cyclodextrin or derivatives of the latter (PCT/EP95/02656).

According to the invention, the active ingredients can also be encapsulated with liposomes.

In addition, the invention is to be explained by the figures and examples below. Here:

FIG. 1 shows the action of test substances on the expression of an NF_{K}b-controlled reporter gene in an ERα-positive cell.

FIG. 2 shows the action of test substances on the expression of an NF_{K}b-controlled reporter gene in an ERβ-positive cell.

FIGS. 3 and 4 show the action of test substances or combinations of test substances on the expression of an NF_{K}b-controlled gene in an ERα-positive cell and an ERβ-positive cell.

### EXAMPLES

Methodology

Antiestrogenicity in vitro

The antiestrogenic action of SERMs is determined by transactivation tests in MVLN cells. In this case, these are MCF-7 breast cancer cells that were transfixed in a stable manner with a Vitellogenin-ERE-luciferase reporter gene (Demirpence et al. (1993), J. Steroid Biochem. Mo. Biol. 46, 355-364).

Estrogen Receptor Binding Studies

The binding affinity of the new selective estrogens (ERβ-ligands) and SERMs was tested in competitive experiments with use of 3H-estradiol as a ligand in estrogen receptor preparations of rat prostates and rat uteri. The preparation of the prostate cytosol and the estrogen receptor test with the prostate cytosol was performed as described by Testas et al. (1981) (Testas, J. et al. (1981), Endocrinology 109, 1287-1289).

The preparation of the rat uterus cytosol as well as the receptor test with the ER-containing cytosol were basically performed as described by Stack and Gorski, 1985 (Stack, Gorski 1985, Endocrinology 117, 2024-2032) with some modifications as described by Fuhrmann et al. (1995) (Fuhrmann, U. et al. (1995), Contraception 51, 45-52).

The ERβ-ligands that are claimed in this patent for the application in the combination preparation have a higher binding affinity to estrogen receptors of rat prostates than rat uteri. In this case, it is assumed that ERβ predominates in the rat prostates over ERα, and ERα predominates in the rat uteri over ERβ. In accordance with this, we find that the ratio of the binding to prostate and uterus receptors corresponds qualitatively to the quotient of relative binding affinity (RBA) to human ERβ and rat ERα (according to Kuiper et al. (1996), Endocrinology 138, 863-870).

In addition, the predictability of the “prostate-ER versus uterus-ER-test system” with respect to tissue-selective action was confirmed by in vivo studies. Substances with a preference for prostate-ER are dissociated in vivo with respect to bone and uterus action.

Repression JF_{K}b-Induced Promoters

The reporter gene assay was performed in U2-OS human osteosarcoma cells as described (Fritzemeier, Hegele-Hartung (1999), Handbook of Pharmacol., Oettel, Schillinger Editors 135/II, 21, 1-94). The cells were transfixed in a transient manner with a reporter gene, which was under the control of a promoter that contains an NF_{K}b-binding site. In addition, the cells were transfixed with expression vectors for hERα and/or hERβ.

1. Pharmaceutical combination preparation that comprises
(a) at least one agonist that is selective for the estrogen receptor β (ERβ), and
(b) at least one antiestrogen and/or at least one selective estrogen receptor modulator (SERM).

2. Preparation according to claim 1, wherein it contains components (a) and (b) in a common dispensing form.

3. Preparation according to claim 1, wherein it contains components (a) and (b) in dispensing forms that are separate in each case.

4. Preparation according to one of claims 1 to 3, wherein the ERβ antagonist (a) is selected from 3,16-dihydroxyestra-1,3,5(10)-triene derivatives, 8α-H, 9β-H, 10α-H, 13α-H, 14β-H-gonane derivatives, preferably derived from ent-13-alkylgonane, 8β-substituted estra-1,3,5(10)-triene derivatives and gona-1,3,5(10)-triene derivatives.

5. Preparation according to one of claims 1 to 4, wherein component (b) contains an antagonist that is selective for the estrogen receptor α (ERα), in particular a peripherally-selective ERα-selective antagonist.

6. Preparation according to one of claims 1 to 4, wherein component (b) contains a SERM that is selective for ERα.

7. Use of a combination preparation according to one of claims 1 to 6 for a tissue-selective or organ-selective estrogen therapy.

8. Use according to claim 7 for the prophylaxis or treatment of perimenopausal and postmenopausal symptoms, for hormone substitution, for prophylaxis or treatment of hormone-deficiency-induced symptoms, in particular in the case of ovarian dysfunction, for prophylaxis and treatment of hormone-deficiency-induced bone mass loss and osteoporosis, for prophylaxis and treatment of cardiovascular and vascular diseases, for prophylaxis and treatment of hormone-deficiency-induced and neurodegenerative diseases, for prophylaxis and treatment of hormone-deficiency-induced impairments of memory and learning capacity, and for prophylaxis and treatment of diseases of the immune system.
